(19) United States
(12) Patent Application Publication
(10) Pub. No.: US 2009/0024122 A1
(43) Pub. Date: Jan. 22, 2009

(54) Endoscopic-surgery apparatus for argon-plasma coagulation (apc)

(76) Inventor: Klaus Fischer, Nagold (DE)

Correspondence Address: DICKSTEIN SHAPIRO LLP, 1825 EYE STREET NW, Washington, DC, 20006-5403, US

(21) Appl. No.: 11/920,059

(22) PCT Filed: May 2, 2006
(86) PCT No.: PCT/EP2006/004100

§ 371 Date: Nov. 8, 2007

(30) Foreign Application Priority Data
May 9, 2005 (DE) 102005021304.9

(51) Int. Cl. A61B 18/14

(52) U.S. Cl. 606/40; 600/104

(57) ABSTRACT

The invention relates to an endoscopic-surgery apparatus for argon-plasma coagulation (APC) to treat biological tissue. The surgery apparatus comprises a tube, a tubular probe or similar working device that can be inserted into an endoscope, with a first channel such that by way of the first channel, as a first working means, there can be guided to the tissue an electrode connected to an HF generator in order to generate a high-frequency current, as well as argon or a similar inert gas. This endoscopic-surgery apparatus is further developed in order to simplify endoscopic interventions employing argon-plasma coagulation, while simultaneously increasing the efficiency of a treatment. To this end, the working device comprises at least one second channel through which at least one second working means is guided to the tissue.

### FIELD OF THE INVENTION

The invention relates to an endoscopic-surgery apparatus for argon-plasma coagulation (APC).

### BACKGROUND OF THE INVENTION

High-frequency surgery, which employs techniques including argon-plasma coagulation, has been applied for many years in both human and veterinary medicine in order to coagulate and/or to cut biological tissue. In such procedures, suitable electro-surgical instruments are used to pass a high-frequency electrical current through the tissue to be treated, so that this tissue is altered by protein coagulation and dehydration. Therefore vessels can be closed and bleeding stanched by means of a coagulation process. A cutting process that follows the coagulation process then enables already coagulated tissue to be completely transected.

The method of argon-plasma coagulation enables non-contact coagulation of tissue and achieves effective stanching of blood and devitalization of tissue. In this kind of coagulation, an inert working gas, such as argon, is guided to the tissue to be treated by way of gas-delivery devices which are part of an argon-plasma-coagulation instrument designed to determine the argon dosage and monitor for errors. Such gas-delivery devices comprise an APC probe that also contains an electrode to supply a high-frequency (HF) current to the distal end of the probe. The electrode is disposed in the probe in such a way that it does not make contact with the tissue during the treatment. By means of the working gas and the high-frequency current, a plasma is generated between the distal end of the probe and the tissue. The application of current to the tissue is brought about by way of the plasma stream. The term “plasma stream” here signifies a coagulation current that flows along a path resembling a flexible tube. Adhesion of the tissue to the electrode is thus prevented. Furthermore, argon-plasma coagulation prevents carbonization of the tissue, to a great extent, as well as the formation of smoke and offensive odors.

The technique of argon-plasma coagulation is used for both surgically opened body and minimally invasive operations. In the latter application the probe for delivering the working gas is, for example, pushed through an endoscope that has been inserted through a body opening and into the operation region. A flexible or rigid tube is used as the endoscope. The endoscope preferably comprises several channels and is pushed into the organ to be investigated or into the body cavity. In addition to the APC probe described above, diverse working tools can be guided to the operation region, for example other surgical instruments, by way of the usually multi-lumen endoscope. It is also possible to use the lumens for rinsing, sucking material away or taking a tissue sample. The endoscope further comprises an optical system so that the treatment can be monitored by means of imaging methods.

Because of their multi-lumen design, endoscopes often have a large diameter, so that after the working tools required for a treatment have been guided to the operation region, they are spaced apart from one another by correspondingly large distances. For instance, if a rinsing jet is needed during coagulation of the tissue to be treated, the site where the fluid is introduced to the operation region must be a certain distance away. This is particularly problematic when the course of the operation depends on precise performance of the treatment.

Various interventions often require that the APC probe be advanced out of an outlet opening of the endoscope and into the operation region by different distances, so that, in particular, the endoscope channels used for supplying fluids or sucking away vapor or tissue liquids cannot follow. This makes the operation considerably more difficult.

The patent EP 0 957 793 B1, for example, discloses a flexible argon-plasma endoscopy coagulator with a tube that can be inserted into an endoscope for guiding an electrode and a gas to a tissue that is to be treated. At a distal end of the tube is provided a device that in addition to a plasma stream or plasma creates a protective-gas atmosphere that is favorable to the APC. Thus, for example, a ceramic end piece constructed as a diffuser is inserted into the distal end of the tube in order to produce turbulence in the gas current and thus build up a “cloud” of argon around the plasma stream. However, the inserted end piece requires considerable effort to use and cannot readily be removed, if necessary, during an operation.

It is therefore the objective of the present invention to develop an apparatus for endoscopic surgery of the kind described above, in such a way as to simplify endoscopic interventions by means of argon-plasma coagulation, while simultaneously increasing the efficiency of a treatment.

### BRIEF DESCRIPTION OF THE DRAWINGS

The invention is described with reference to exemplary embodiments, which are explained in greater detail with reference to the drawings, wherein:

FIG. 1 illustrates an endoscopic-surgery apparatus with a probe according to a first preferred embodiment, while the probe is in use;

FIG. 2 illustrates a cross-sectional side view of a distal end of the probe according to a second preferred embodiment;

FIG. 3 illustrates a cross-sectional side view of the distal end of the probe according to a third preferred embodiment;

FIG. 4 illustrates a cross-sectional side view of the distal end of the probe according to a fourth preferred embodiment;

FIG. 5 illustrates a schematic end-face view of the distal end of the probe according to the second preferred embodiment;

FIG. 6 illustrates a schematic end-face view of the distal end of the probe according to a fifth preferred embodiment;

FIG. 7 illustrates a schematic end-face view of the distal end of the probe according to the first preferred embodiment; and

FIG. 8 illustrates a distal end of a probe such as is known from the state of the art.

### DETAILED DESCRIPTION OF THE INVENTION

The objectives of the present invention are achieved by an endoscopic-surgery apparatus for argon-plasma coagulation (APC) to treat biological tissue that comprises a tube and a tubular probe or similar working device. The working device can be inserted into an endoscope and has a first channel through which, as a first working means, an electrode connected to an HF generator to generate a high-frequency electrical current can be guided to the tissue, as well as argon or a similar inert gas. The working device also has at least a second channel to guide at least a second working means to the tissue.

According to embodiments of the invention, other working means can be guided to the operation region and can be employed at the operation region in the immediate vicinity of a plasma stream or plasma produced at a distal end of the probe in addition to the working device of the endoscopic-surgery apparatus (e.g., the APC probe). For this purpose both the first and the second channel each comprise at least one outlet opening at the distal end of the probe.

In a first preferred embodiment, the second channel is constructed so that a fluid can be supplied to or carried away from the tissue as the second working means. This presents the opportunity of supplying another current of inert gas, such as argon, as a “pre-flow” that can be started prior to activation of the working flow needed to generate the plasma stream and before the HF current from the APC probe is switched on.

The delivery of argon to the operation region before activation of the HF current is known, for example, from the document DE 101 29 685 A1. In that case, before the APC is carried out a specified amount of argon flows out of a single-lumen APC probe (argon pre-flow) in order to sufficiently reduce the concentration of combustible gases (e.g., oxygen or carbon monoxide) at the operation region such that ignition of these gases or this mixture of gases is not possible.

On the other hand, if an additional gas current, e.g., an argon current, is continuously, or in some circumstances as a series of pulses, supplied to the operation region even during the actual APC, then an argon atmosphere is maintained as a protective atmosphere in the immediate surroundings and the above-mentioned combustible gases are permanently displaced, from the operation region. The protective flow that is to be maintained during coagulation, along with the working flow, can be introduced above the coagulation region by way of the second working channel of the APC, or in some cases by way of several additional working channels, so that an atmosphere that assists the APC can thereby be precisely created. This atmosphere can thus be adjusted independently of the position of the endoscope with reference to the probe. Furthermore, by way of the second channel, the gas concentration can be arbitrarily varied. Penetration of liquid into the probe is also prevented by the additional gas flow.

It is also possible to deliver other fluids to the tissue that is to be treated, by way of another channel in the APC probe. For example, rinsing liquids or gases can be guided extremely precisely to the coagulation region, or also sucked away from that region. A treatment with medication can also be done by way of an additional channel in the probe.

Preferably, the second channel comprises a nozzle device to distribute the supplied fluid at a distal end of the channel. Thus it is possible, for example, to atomize a liquid such as a NaCl solution and use it for wetting the tissue to be treated in order to achieve improved conductivity of the tissue. In addition to a cooling action of the liquid, a carbonization effect is distinctly reduced. When a conductive liquid is used, the current density during the HF application can be reduced, so that so-called hot spots appear only occasionally. This is particularly advantageous for thin layers of tissue, in which a homogeneous and limited depth control is desired.

One solution in accordance with the invention provides that the second channel be constructed as a second working means that allows equilibration of a difference between the pressure prevailing in an operation region and the pressure in the surroundings. This is required when an additional gas current, as described above, is guided to the operation region or when gas is needed to expand a body cavity, such as the esophagus, so that its surface is “pulled smooth”.

If the second channel is constructed so that a surgical instrument can be guided to the tissue as the second working means, the additional instrument can operate at precisely the desired place. Here, again, employment of the instrument is independent of the position of the probe with reference to the endoscope.

An especially simple way of introducing the second working means, in particular for generating the protective atmosphere, is enabled by a substantially coaxial arrangement of the second channel with respect to the first channel.

Alternatively it is possible for the second channel to run parallel to the first channel, which is suitable in particular for introducing an additional surgical instrument or supplying a rinsing fluid.

In order to introduce additional inert gas to the operation region, even during the APC, at least one first outlet opening is disposed at the distal end of the second channel, in a side wall or jacket surface at the distal end of the working device, i.e. the APC probe. For example, when the channels are arranged parallel to one another it is possible to provide only one such outlet that opens into the side wall. In the case of a coaxial arrangement of the at least two channels in the probe, several outlet openings of the second channel can be disposed in the outer jacket surface of the probe. The laterally disposed outlet openings of the second channel are, in this case, as a rule, positioned before the outlet opening of the first channel, where “before” refers to an axial direction of extent of the probe, in the direction towards its distal end. Thus the protective atmosphere, in particular when the channels are coaxially arranged, can be produced extremely reliably, because it is ensured that a distal end of the first channel and hence also the plasma stream during the APC are completely within the protective atmosphere. The coagulation current independently finds its own “ideal” path through the atmosphere.

In another preferred embodiment at least a second outlet opening is provided at the distal end of the second channel, at a distal end face of the working device. In the case of coaxial arrangement of the channels, the plasma stream can thus be precisely enveloped by an additional gas current simultaneously supplied. A parallel arrangement of the channels, and hence a side-by-side arrangement of the outlet openings, enables other surgical instruments to be guided directly to the operation region, by simple means. The adjustment of a protective flow prior to and/or during the APC can likewise thereby be performed by simple means.

Preferably, the outlet opening of the second channel disposed in the side wall and/or at the distal end face of the working device is positioned, with reference to the axial direction of extent of the working device, in the direction towards its distal end, before the outlet opening of the first channel. That is, the first channel extends, for example, beyond the second channel. Thus the protective atmosphere can be built up around the plasma stream extremely reliably by the protective flow, in particular when the channels are coaxially arranged, because as explained above it is ensured that the distal end of the first channel and hence also the plasma stream needed for the APC are completely within the protective atmosphere.

Preferably, the working device is constructed with three channels arranged substantially parallel to one another, such that the outlet opening of the second channel and an outlet opening of the third channel are disposed, with reference to the axial direction of extent of the working device in the direction towards its distal end, before the outlet opening of the first channel. The outlet opening of the second channel and the outlet opening of the third channel are positioned at the same height with reference to the direction of extent. Here, again, the first channel extends beyond the second and third channels, so that by simple means the plasma stream created by way of the first channel can be enveloped by the protective atmosphere generated by way of the second and/or the third channel. That is, the ionizable gas not only fills the space between the outlet opening of the probe and the tissue to be treated, as would be the case for instance with a single-lumen probe, but also fills a larger volume, through which the coagulation current can find its way. When there are three or even more channels in the APC probe, in addition to the protective flow it is also possible to employ an additional surgical instrument during the APC. Alternatively, the third channel may serve the purpose of pressure equilibration, as previously described. Fundamentally, all outlet openings can be disposed at the same height or can be at different heights. The channels—regardless of their number—can have different diameters and different lengths, or they can be uniformly constructed.

In the following description, the invention is described with reference to the drawings, wherein the same reference numerals are used to describe identical parts or parts with identical actions.

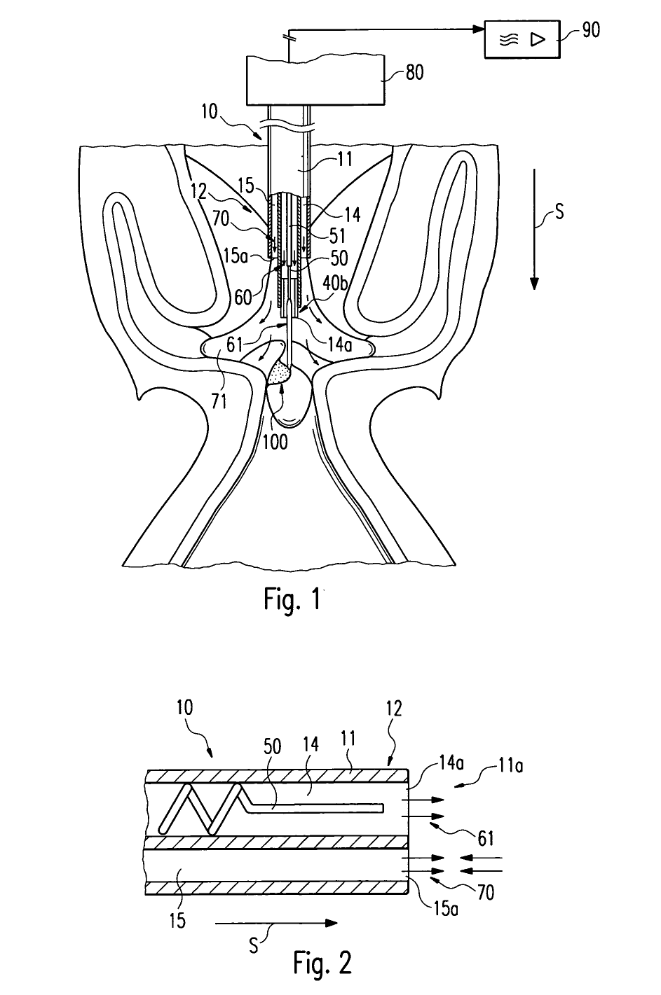

FIG. 1 shows an endoscopic-surgery apparatus 10 with a working device 11 according to a first preferred embodiment, while the device is in use. The working device 11 is constructed as a probe for argon-plasma coagulation (APC). By way of an endoscope 80, the probe 11 has been guided to a tissue 100 which is to be treated, in this case in the region of the vocal folds of a patient. The probe 11 comprises a first working channel 14 for the argon-plasma coagulation and a second working channel 15 disposed coaxially thereto. At the distal end of each of the channels 14 and 15 is an outlet opening 14a or 15a, respectively. An electrode 50 supplies a high-frequency current to a distal end 12 of the probe 11, and thus to the tissue 100 that is to be treated. The electrode 50 is disposed within the first working channel 14. The electrode 50 is connected by way of current-delivery devices 51 to an HF generator 90 for producing a high-frequency voltage. During the APC an inert gas 60, preferably argon, flows around the electrode 50 so that, due to an interaction between the HF current and the gas, a plasma 61 is produced. Within the probe 11 the electrode 50 opens into a nozzle device 40b, so as to obtain a plasma stream 61 that is as well targeted as possible. By way of the plasma stream 61, the HF current can be guided to the tissue 100, so that the tissue 100 is coagulated.

By way of the second channel 15, which is disposed coaxially with respect to the first channel 14, another gas flow 70 may be directed to the operation region. This can occur prior to ionization or during the argon-plasma coagulation. This gas flow 70, preferably a current of argon, encloses the plasma stream 61 so that an envelope 71 of inert gas is built up by the protective flow 70 in the immediate surroundings of the plasma stream 61. That is, the ionizable gas fills not only the space between the outlet opening of the probe and the tissue to be treated, as would be the case for example with a single-lumen probe; instead, it fills a larger volume, through which the coagulation current can find its way. The gas envelope 71 acts as a protective atmosphere, displacing reactive gases such as oxygen or carbon monoxide from the operation region, so that ignition of these gases in association with the plasma stream 61, which would be dangerous to the patient, is prevented.

The outlet opening 15a of the second channel 15 in this exemplary embodiment is disposed, with reference to an axial direction S of extent of the probe 11, towards the distal end 12 of the probe 11 and before the outlet opening 14a of the first channel 14. That is, the first channel 14 projects out of the second channel 15. Thus the protective atmosphere 71 can be built up with extreme reliability, because it is ensured that the distal end of the first channel 14 and hence the plasma stream 61 are situated completely within the protective atmosphere 71.

Because the second channel 15 for supplying additional argon to the operation region is disposed within the APC probe 11, the formation of the argon cloud that envelops the plasma stream 61, i.e. the protective atmosphere 71, is independent of the position of the probe 11 in relation to the endoscope 80. Furthermore, the additional argon flow can be arbitrarily turned on and off, depending on the extent to which the protective flow 70 is desired.

FIG. 2 shows a sectional side view of the distal end 12 of the probe 11 according to a second preferred embodiment. Here the first channel 14, which contains the electrode 50, and the second channel 15 are disposed substantially parallel to one another within the APC probe 11 rather than in a coaxial configuration. With this embodiment, again, the protective flow 70 can be guided to the operation region without problems. The second channel 15, however, can also be used to suck away surplus gas or other fluids that are present in the operation region and/or, if necessary, to bring about pressure equilibration. It is also possible to supply a liquid for wetting the tissue 100 that is to be treated by way of the second channel 15, as was the case with the coaxial arrangement according to FIG. 1. In this way, the electrical conductivity is increased to produce a better coagulation result. When the channels 14, 15 are side by side, it is also possible for another surgical instrument (not shown) to be introduced into the operation region in order to assist the APC. The electrode 50 comprises a region in helical form, to enable the electrode 50 to be braced within the first channel 14 and thereby retained in the correct position. FIG. 5 shows schematically an end face 11a at the distal end 12 of the probe 11 according to this embodiment, in which the electrode is not represented. The channels 14, 15 here are shown as having equal diameters. Depending on the particular application, the channels may also have diameters that are different from one another.

Alternatively, it would be possible for the outlet opening of the second channel, when in the parallel arrangement, to be disposed before or after the outlet opening of the first channel, towards the distal end of the probe in the direction of its extent, so that one channel extends further than the other.

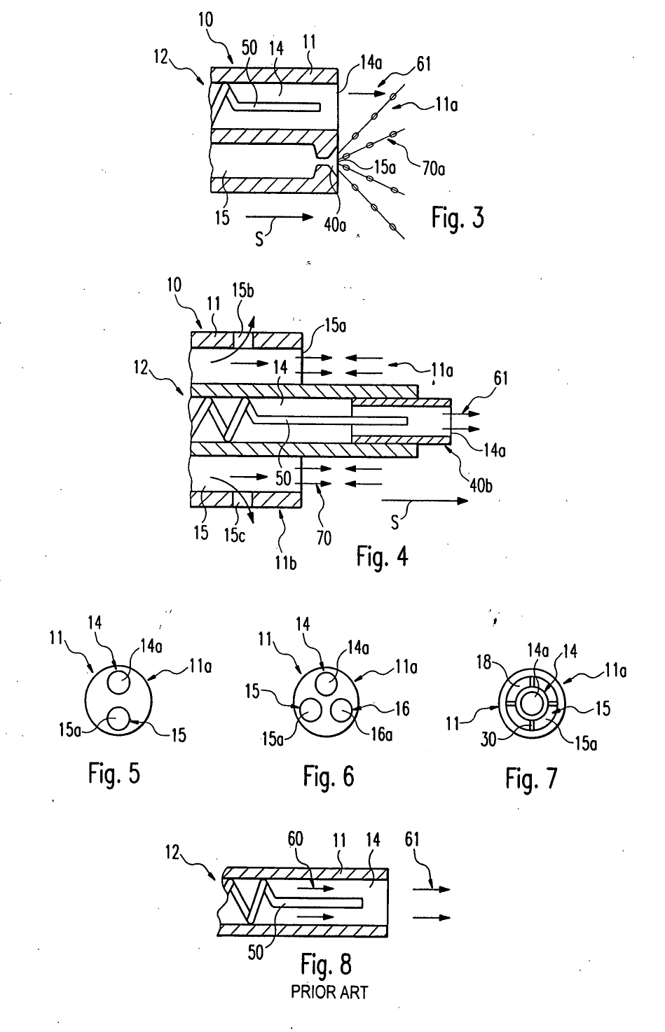

FIG. 3 shows a sectional side view of the distal end 12 of the probe 11 according to a third preferred embodiment. The embodiment of FIG. 3 corresponds substantially to that shown in FIG. 2, however the second channel 15 additionally comprises a nozzle device 40a at the outlet opening 15a at the distal end of the channel 15. The nozzle device 40a serves to distribute a fluid 70 (which needs to be delivered in some cases) at the operation region. For instance, the nozzle can be used to atomize a liquid 70a and thereby wet the tissue 100 that is to be treated. The application of the fluid 70a to the tissue 100 improves its conductivity, in case this is necessary, and also cools the tissue and reduces a carbonization effect. When a conductive liquid is used, the current density during the HF application is reduced, so that so-called hot spots occur only occasionally. This is advantageous in particular for thin layers of tissue, in which homogeneous and limited depth control is desired.

FIG. 4 shows a side view of the distal end 12 of the probe 11 according to a fourth preferred embodiment. This embodiment corresponds substantially to that shown in FIG. 1.

Additionally, however, the embodiment according to FIG. 4 comprises at a side wall 11b of the probe 11 (i.e., at its jacket surface at the distal end 12) and outlet openings 15b, 15c for the second channel 15. The outlet openings 15b, 15c can be provided an any desired number. A radially symmetric arrangement of the outlet openings of the second channel 15 with respect to a long axis, considered to lie in the axial direction of extent S of the probe 11, ensures that a large volume of the protective atmosphere 71 will reliably enclose the plasma stream 61 that emerges by way of the first channel 14.

FIGS. 6 and 7 each show schematic end-face views of the distal end 12 of the probe 11 according to a fifth preferred embodiment (FIG. 6) or according to the first embodiment (FIG. 7). The three-lumen probe 11, illustrated schematically in FIG. 6 by the outlet openings 14a, 15a, 16a of the first, the second and a third channel 14, 15, 16, makes it possible, for example, for the protective flow 70 to be employed while an additional surgical instrument (not shown) is simultaneously guided to the operation region. In FIG. 7 the coaxial arrangement of the first channel 14 and the second channel 15 within the APC probe 11 is shown. Here supporting devices 30 are provided, which enable the first channel 14 to be spaced apart from the second channel 15.

It is fundamentally also possible to construct the first channel in such a way that its outlet opening is disposed at the side wall of the APC probe.

FIG. 8 shows the distal end 12 of a probe 11 known from the state of the art, which comprises a channel 14 to supply the gas 60 required for the APC, e.g. argon. There is also disposed in the channel 14 an electrode 50, so that by way of a plasma 61 the high-frequency current can be guided to a tissue to be treated.

At this juncture it should be pointed out that all of the parts described above are claimed as essential to the invention, individually or in any combination, in particular the details illustrated in the drawings. Modifications thereof are familiar to a person skilled in the art.

1. An endoscopic-surgery apparatus for argon-plasma coagulation (APC) to treat biological tissue, comprising:
a working device configured to be inserted into an endoscope, the working device comprising a first channel,
such that an electrode connected to a high-frequency generator, in order to generate a high-frequency current, is guided to the tissue by way of the first channel, as a first working means, and argon or a similar inert gas is also guided to the tissue by way of the first channel, and,
wherein the working device further comprises at least one second channel, through which at least one second working means is guided to the tissue.

2. The endoscopic-surgery apparatus according to claim 1, wherein the second channel is constructed so that a fluid is supplied to the tissue or conducted away from the tissue, as the second working means.

3. The endoscopic-surgery apparatus according to claim 2, wherein the second channel is constructed so that as the fluid is argon or a similar inert gas or a liquid and is guided to or away from the tissue.

4. The endoscopic-surgery apparatus according to claim 1, wherein the second working means is a fluid and the second channel comprises a nozzle device at a distal end for distributing the fluid.

5. The endoscopic-surgery apparatus according to claim 1, wherein the second channel is constructed such that the second working means equilibrates a pressure difference between a pressure prevailing in an operation region and a surrounding pressure.

6. The endoscopic-surgery apparatus according to claim 1, wherein the second channel is constructed so that a surgical instrument is guided to the tissue as the second working means.

7. The endoscopic-surgery apparatus according to claim 1, wherein the second channel is disposed essentially coaxially with the first channel.

8. The endoscopic-surgery apparatus according to claim 1, wherein the second channel is disposed substantially parallel to the first channel.

9. The endoscopic-surgery apparatus according to claim 1, wherein at least one first outlet opening is disposed at the distal end of the second channel in a side wall at a distal end of the working device.

10. The endoscopic-surgery apparatus according to claim 9, wherein at least one second outlet opening is disposed at the distal end of the second channel in a distal end face of the working device.

11. The endoscopic-surgery apparatus according to claim 10, wherein the outlet opening of the second channel disposed in the side wall and/or at the distal end face of the working device is positioned ahead of the outlet opening of the first channel, with reference to an axial extent of the working device in the direction towards its distal end.

12. The endoscopic-surgery apparatus according to claim 11, wherein the working device is constructed with three channels disposed substantially parallel to one another, such that the outlet opening of the second channel and an outlet opening of the third channel are positioned ahead of the outlet opening of the first channel, with reference to the axial direction of extent of the working device in the direction towards its distal end, the outlet opening of the second channel and the outlet opening of the third channel being situated at the same height with reference to the direction of extent.

13. An endoscopic-surgery apparatus, comprising:
a working device comprising a first channel and a second channel,
wherein the first channel is configured to guide a first working means to a biological tissue to be treated by argon-plasma coagulation, and wherein the second channel is configured to guide a second working means to the biological tissue.

14. The endoscopic-surgery apparatus of claim 13, wherein the first working means is an electrode configured to generate a high-frequency current.

15. The endoscopic-surgery apparatus of claim 14, wherein the first channel is further configured to guide an inert gas to the biological tissue, the inert gas forming a plasma stream by which the high-frequency current is guided to the biological tissue.

16. The endoscopic-surgery apparatus of claim 15, wherein the second working means is an inert gas which forms a protective atmosphere surrounding the plasma stream.

17. The endoscopic-surgery apparatus of claim 13, further comprising a third channel configured to guide a third working means to the biological tissue, wherein one of the second working means and the third working means is a surgical instrument

18. A surgical instrument configured for insertion into an endoscope, the surgical instrument comprising:
a working device comprising at least a first channel and a second channel,
wherein the first channel is configured to guide an electrode and a first inert gas stream to a biological tissue to be treated, the electrode being configured to generate a high-frequency current, and the first inert gas stream forming a plasma stream upon interaction with the electrode,
wherein the second channel is configured to guide a second inert gas stream to the biological tissue to be treated, and
wherein the first channel extends past the second channel, in a distal direction of the working device, such that the second inert gas fully surrounds a distal end of the first channel and forms a protective atmosphere around the electrode and plasma stream.

19. The surgical instrument of claim 18, wherein at least one of the first inert gas stream and the second inert gas stream comprises argon.

20. The surgical instrument of claim 18, wherein the second channel is disposed to be one of essentially coaxially or substantially parallel with respect to the first channel.
